(19) United States
(12) Patent Application Publication
(10) Pub. No.: US 2023/0000737 A1
(43) Pub. Date: Jan. 5, 2023

(54) DRY COMPOSITIONS, EMULSIONS AND/OR FLUIDS FOR CHEMICAL AND PHYSICAL SUN PROTECTION AND USE THEREOF

(71) Applicant: OMYA INTERNATIONAL AG, Oftringen (CH)

(72) Inventors: Jamal FTOUNI, Zofingen (CH); Fabrizio ORLANDO, Würenlos (CH); Joachim SCHOELKOPF, Oberkulm (CH)

(73) Assignee: OMYA INTERNATIONAL AG, Oftringen (CH)

(21) Appl. No.: 17/756,678

(22) PCT Filed: Dec. 7, 2020
(86) PCT No.: PCT/EP2020/084936

§ 371 Date: May 31, 2022

(30) Foreign Application Priority Data
Dec. 10, 2019 (EP) 19214857.5

(51) Int. Cl. A61K 8/19; A61K 8/02; A61K 8/9789; A61K 8/06; A01N 3/00; A61Q 17/04

(52) U.S. Cl. CPC A61K 8/19; A61K 8/0241; A61K 8/9789; A61K 8/06; A01N 3/00; A61Q 17/04

(57) ABSTRACT

The present invention refers to a dry composition for chemical and physical sun protection, the composition comprising a) at least one calcium carbonate, and b) from 0.1 wt.-% to 100 wt.-%, based on the dry weight of the at least one calcium carbonate of step a) of at least one lignin. Furthermore, the present invention refers to a fluid composition comprising the inventive dry composition as well as to an emulsion comprising the inventive dry composition. The present invention also refers to the use of the inventive compositions for sun protection of plants and parts thereof as well as the use of the inventive emulsion for chemical and physical sun protection in a cosmetic formulation.

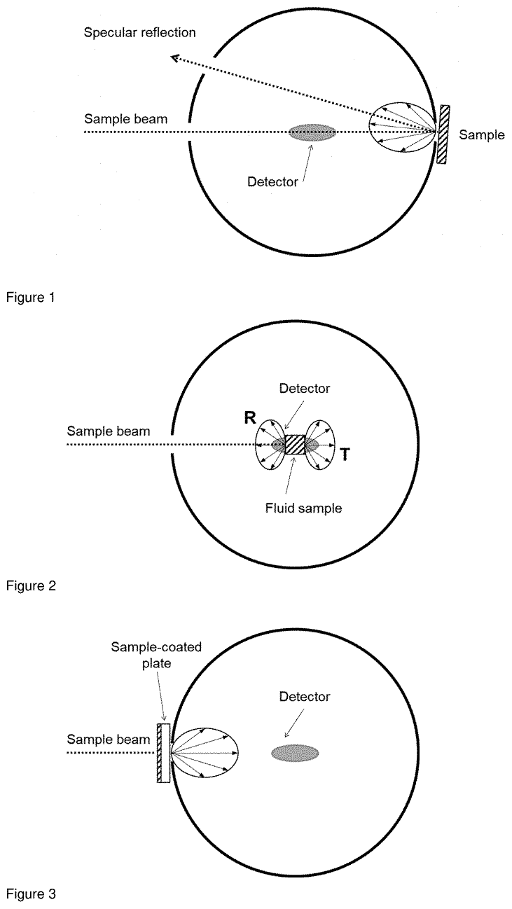

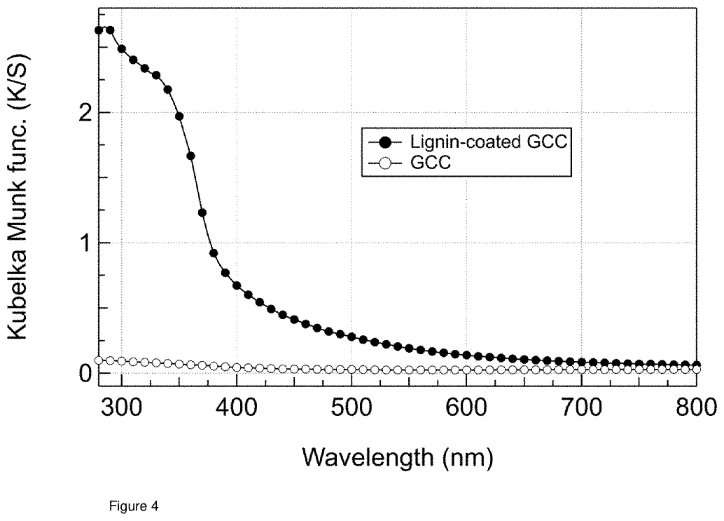

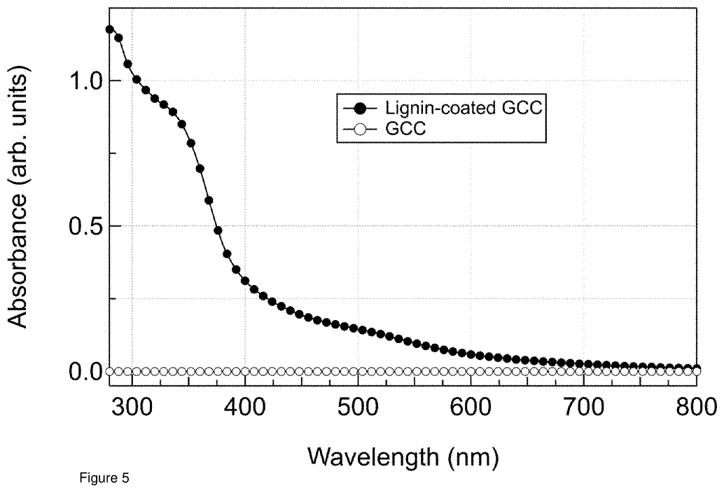

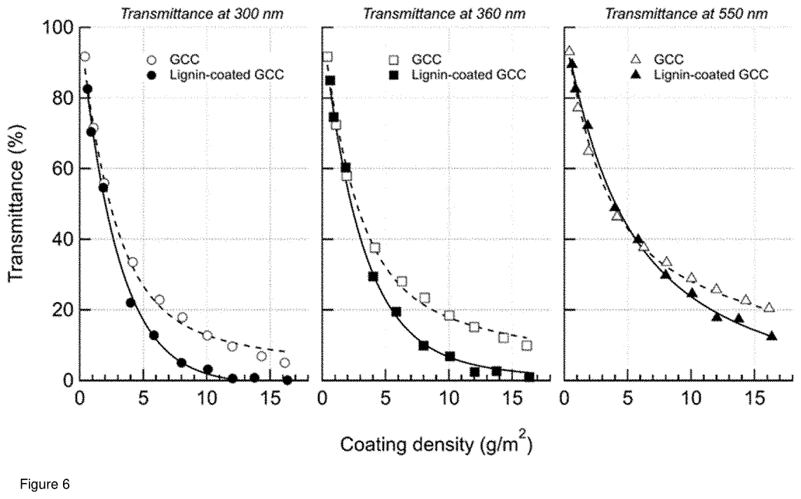

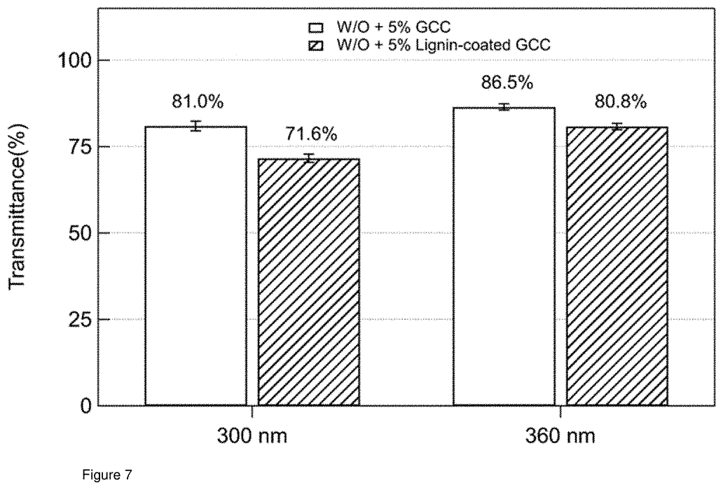

The present invention refers to a dry composition for chemical and physical sun protection, the composition comprising a) at least one calcium carbonate, and b) from 0.1 wt.-% to 100 wt.-%, based on the dry weight of the at least one calcium carbonate of step a) of at least one lignin. Furthermore, the present invention refers to a fluid composition comprising the inventive dry composition as well as to an emulsion comprising the inventive dry composition. The present invention also refers to the use of the inventive compositions for sun protection of plants and parts thereof as well as the use of the inventive emulsion for chemical and physical sun protection in a cosmetic formulation.

It is well known that the sunlight energy and mainly the ultraviolet portion of the sun's spectrum has a damaging effect on living cells, especially on plants and parts thereof as well as on the human skin. In particular, the ultraviolet B (UV-B) radiation which ranges from 280 to 320 nm and the ultraviolet A (UV-A) radiation which ranges from >320 to 400 nm both cause sunburn to plants and parts thereof. Especially, when the shaded air temperature is above a temperature of ca. 30° C., plants and parts thereof and especially fruits can be damaged due to sunburn. The consequence of such a “fruit sunburn” is a damage of the fruits and a decrease of the total production yield, causing an economic impact, mainly in hot summer seasons.

Furthermore, the ultraviolet A (UV-A) and ultraviolet B (UV-B) radiation have a damaging effect on the human DNA by the formation of free radicals and other reactive species developed through phototoxic reactions in the epidermis and dermis of the skin. As a consequence, the UV-A and UV-B radiation is now considered as a main factor in the development of chronic light-induced alterations such as premature ageing of the skin and development of skin cancer.

Therefore, it is becoming increasingly important to protect living cells from sunburn. Especially, to protect at least the part of the plants and parts thereof and especially, the fruits which are exposed to sun light. Also the human skin which is exposed to sun light has to be protected against UV-B as well as UV-A radiation. In the art several attempts have been made to provide such UV protection.

One attempt to protect the plants from sunburn is by covering up the living cells from the sunlight by opaque materials such as foils or fabrics. For example, CN203538002 refers to a pomegranate cultivation bag capable of preventing sunburn, wherein one side of the pomegranate cultivation bag is reflective, and the other side is transparent. During a high temperature season, the pomegranate cultivation bag is sleeved over a pomegranate with the reflective side facing the sun to reflect most of sun light in the noon to prevent sunburn of the pomegranate, and partial sun light reflected by a reflective plastic film irradiates on the leaves at the shady side of a fruit tree to increase the photosynthesis of the entire orchard. However, such a procedure is complex, time-consuming and labor-intensive, since every plant or fruit has to be treated individually by hand.

Another option to reduce the probability of sunburn is the using of mineral particles, usually clay or calcium carbonate, which form a film on the plants and parts thereof or on the human skin and protect the living cells by reflecting or absorbing the damaging UV radiation. Therefore, these compounds are known as physical sun protecting agents. Such sunscreen compositions for applications to plants are known, for example from US 2012/0052187 A1 that refers to a sunscreen composition comprising Titanium Dioxide (TiO2), Zinc Oxide (ZnO), Silicon Dioxide (SiO2), a surfactant, wetting agent, dispersant (SWD) and water. The composition forms a suspension concentrate that when diluted in water provides a solution that provides uniform coverage using conventional spraying equipment.

WO 2010/008476 A1 refers to a method for increasing a yield of a crop including controlling plant tissue stress by at least partially coating a plant tissue with a composition comprising an agricultural sunscreen formulation and a second agricultural chemical. The agricultural sunscreen formulation may comprise 40 to 80 wt.-% calcium carbonate, 1 to 5 wt.-% bicarbonate and 15 to 59 wt.-% water.

US2016037772 A1 refers to a method for treating a plant in order to increase tolerance to abiotic stress in the plant or to reduce a consequence of abiotic stress in the plant. Sunblocks can be added such as kaolin or calcium carbonate.

US2017333301 A1 refers to a water-in-oil emulsion sunscreen cosmetic comprising 6 to 40 mass % of a UV protective agent; an organic-modified clay mineral; an oil-phase-thickening agent, and a silicone-based surfactant having an HLB of less than 8. The UV-protective agent may be particles of metal oxides such as zinc oxide, titanium oxide, iron oxide, cerium oxide, and tungsten oxide.

Inorganic UV filters such as zinc oxide and titanium dioxide are photostable and give a broad spectrum protection covering UV-A and UV-B by blocking the UV light. So-called mineral-only sunscreen formulations, i.e. sunscreen formulations comprising solely inorganic UV filter materials such as zinc oxide and/or titanium dioxide were long time especially recommended for people with sensitive skin as well as for babies and toddlers. However, the inorganic UV filters are mostly used in the form of nano particles and it is speculated recently that these nano particles might have harmful effects on the nature and the human skin.

The article “Encapsulating TiO2 in Lignin-Based Colloidal Spheres for High Sunscreen Performance and weak Photocatalytic Activity” YuanYuan Li et al., ACS Sustainable Chemistry and Engineering, vol. 7, no. 6, 18 Feb. 2019 refers to nano TiO2 particles that are well trapped in lignin colloidal spheres. However, as already set out above, TiO2 nano particles might have harmful effects on the nature and the human skin. Furthermore, colloidal spheres show different chemical performance than, for example, particles that coated with lignin on the particles.

The article “Lignin coating to quench photocatalytic activity of titanium dioxide nanoparticles for potential skin care applications”, M. Morsella at al., RSC Advances, vol. 5, no. 71, 1 Jan. 2015 refers to lignin coating to quench photocatalytic activity of titanium dioxide nanoparticles for potential skin care applications. However, as already set out above, TiO2 nano particles might have harmful effects on the nature and the human skin. Furthermore, this document refers to the photocatalytic activity which is different to the UV activity.

Another option to reduce the probability of sunburn is the using of organic UV filters. Organic UV filters are often transparent or translucent. Such filters provide chemical sun protection by absorbing the UV-A and/or UV-B radiation. Organic UV filters can be classified in synthetically produced UV filters and natural occurring UV filters. Synthetically produced UV filters do not occur in nature but are produced synthetically whereas natural occurring UV filters occur in nature or are produced from natural occurring compounds.

Synthetically produced UV filters such as, for example, octocrylene can be designed such that they perfectly match with the respective system or composition they are blended with. Additionally, such filters can be customized for the individual application. However, synthetic organic UV filters, for example, such as octocrylene are subject to continuously increasing concerns especially because they are suspected to be a possible cause of skin irritations and allergies in sensitive persons and their potentially harmful effect on the environment.

Therefore, there is a continuous need in the art for adequate formulations or compositions providing sufficient or improved UV-B and/or UV-A protection to living cells especially to plants and parts thereof as well as to the human skin.

Thus, it is an objective of the present invention to provide a composition for providing chemical and physical sun protection. More precisely, it is an objective of the present invention to provide a composition for providing sufficient or improved UV-B and/or UV-A protection to living cells, especially to plants and parts thereof as well as to human skin by different mechanisms, namely by reflecting the UV-B and/or UV-A radiation as well as by absorbing the UV-B and/or UV-A radiation. A further object of the present invention is that the compounds used in these compositions are non-toxic to humans, do not provide a harmful effect on the environment and preferably are approved by the food act to be used on plants and parts thereof, especially on fruits as well as by the FDA for use in cosmetic applications. Another object of the present invention is that the compositions should be easily and quickly produced, cheap and especially easy to handle. It is especially preferred that the compositions stick to the plants and parts thereof as well as to the human skin and don't get washed off easily by rain or sweat.

These and other objectives of the present invention can be solved by a composition for chemical and physical sun protection as described in the present invention and defined in the claims. Advantageous embodiments of the invention are defined in the corresponding sub-claims.

According to one embodiment of the present invention, a dry composition for chemical and physical sun protection is provided, the composition comprising a) at least one calcium carbonate, and b) from 0.1 wt.-% to 100 wt.-%, based on the dry weight of the at least one calcium carbonate of step a) of at least one lignin.

The inventors surprisingly found out that the foregoing composition provides sufficient or improved chemical and physical sun protection. More precisely, the inventors surprisingly found out that the inventive composition offers two different mechanisms for UV-B and/or UV-A protection to living cells, especially to plants and parts thereof as well as to human skin by different mechanisms, namely by reflecting the UV-B and/or UV-A radiation as well as by absorbing the UV-B and/or UV-A radiation. The combination of the at least one calcium carbonate and the at least one lignin results in a new and improved UV protection mechanism which is unattainable with the bare minerals and especially with bare calcium carbonate. Furthermore, the compounds used in the inventive composition, namely the calcium carbonate and the lignin are non-toxic to humans, do not provide a harmful effect on the environment. Often these compounds are also approved by the food act to be used on plants and parts thereof, especially on fruits as well as by the FDA for use in cosmetic applications. Furthermore, the inventors surprisingly found that the compositions of the present invention can be easily and quickly produced, are cheap and especially easy to handle. Furthermore, when the composition according to the present invention is used for sun protection of plants and parts thereof, another advantage might be that the composition further provides calcium nutrients to the plants due to the calcium carbonate in the composition.

According to another aspect of the present invention, a fluid composition for chemical and physical sun protection is provided, the fluid composition comprising water and 1 to 100 wt.-% of the dry composition according to the present invention, based on the weight of the water. Furthermore, another advantage might be that the layer of the composition that forms on the plants and parts thereof, when the fluid composition is used on the plants and parts thereof repels also insects.

According to another aspect of the present invention, an emulsion for chemical and physical sun protection is provided, the emulsion comprising a water in oil or oil in water mixture and 0.1 to 100 wt.-% of the dry composition according to the present invention, based on the weight of the water in oil or oil in water mixture.

According to another aspect of the present invention the compositions according to the present invention are used for sun protection of plants and parts thereof, where the sun protection includes physical and chemical protection.

According to another aspect of the present invention the inventive emulsion is used for chemical and physical sun protection in a cosmetic formulation.

Advantageous embodiments of the above aspects are defined in the corresponding sub-claims.

According to one embodiment of the present invention, the calcium carbonate is selected from the group consisting of ground calcium carbonate (GCC), preferably marble, limestone, chalk, precipitated calcium carbonate (PCC), preferably vaterite, calcite and/or aragonite, and mixtures thereof and most preferably the calcium carbonate is ground calcium carbonate.

According to another embodiment of the present invention, the at least one calcium carbonate has a) a weight median particle size d50 value in the range from 0.05 μm to 20 μm, preferably from 0.25 μm to 10 μm and most preferably from 0.5 μm to 8 μm, and/or b) a top cut (d98) of ≤100 μm, preferably ≤60 μm, more preferably ≤45 μm and most preferably ≤20 μm, and/or c) a specific surface area (BET) of from 0.5 to 100 m2/g, preferably from 0.5 to 50 m2/g, more preferably from 0.5 to 35 m2/g and most preferably from 0.5 to 10 m2/g as measured by the BET nitrogen method.

According to another embodiment of the present invention, the at least one lignin is a water soluble or water insoluble lignin selected from the group consisting of natural lignin, klason lignin, hydrolyzed lignin, milled wood lignin, soda lignin, organosolv lignin, kraft lignin, sulphonated lignin and mixtures thereof, preferably is a water-insoluble lignin selected from the group consisting of klason lignin, kraft lignin and mixtures thereof and most preferably is kraft lignin.

According to another embodiment of the present invention, the at least one lignin is present in the composition in an amount from 1 to 50 wt.-%, based on the dry weight of the at least one calcium carbonate of step a), preferably in an amount of 3 to 30 wt.-% and most preferably in an amount of 5 to 25 wt.-%.

According to another embodiment of the present invention, the composition further comprises an organic solvent, preferably in an amount of 5 to 50 wt.-%, based on the dry weight of the at least one calcium carbonate of step a), more preferably in an amount of 10 to 40 wt.-% and most preferably in an amount of 15 to 35 wt.-% and/or preferably in an amount of 100 to 500 wt.-%, based on the dry weight of the at least one lignin of step b), more preferably in an amount of 150 to 450 wt.-% and most preferably in an amount of 200 to 300 wt.-%.

According to another embodiment of the present invention, the organic solvent is selected from the group consisting of hexane, toluene, methanol, ethanol, dioxane, acetone, dimethyl sulfoxide, dimethylformamide, ethylene glycol, ethylacetate, glycerol, γ-valerolactone, polyethylene glycol, polypropylene glycol and mixtures thereof, more preferably is selected from the group consisting of ethylene glycol, ethylacetate, glycerol, γ-valerolactone, polyethylene glycol, polypropylene glycol and mixtures thereof, and most preferably is γ-valerolactone.

According to another embodiment of the present invention, the at least one lignin is present in the composition in the form of a mixture with or a coating on the at least one calcium carbonate, and preferably is present as a coating on the at least one calcium carbonate.

According to another embodiment of the present invention, the at least one lignin in the coating on the at least one calcium carbonate is a water insoluble lignin, and preferably kraft lignin.

According to another embodiment of the present invention, the fluid composition further comprises a surfactant, preferably selected from the group consisting of monomers and/or co-monomers of acrylic acid, methacrylic acid, itaconic acid, crotonic acid, fumaric acid, maleic anhydride acid, isocrotonic acid, aconitic acid (cis or trans), mesaconic acid, sinapinic acid, undecylenic acid, angelic acid, canellic acid, hydroxyacrylic acid, acrolein, acrylamide, acrylonitrile, dimethylaminoethyl methacrylate, vinylpyrrolidone, vinylcaprolactam, ethylene, propylene, isobutylene, diisobutylene, vinyl acetate, styrene, α-methyl styrene, methyl vinyl ketone, the esters of acrylic and methacrylic acids, organo modified trisiloxane, PEG, polyglycerol-ester, sophorolipid, polyether and mixtures thereof, and most preferably is poly(acrylic acid) and/or poly (methacrylic acid) and/or,

wherein the surfactant is present in the composition in an amount of 0.0001 to 0.2 wt.-%, based on the dry weight of the at least one calcium carbonate of step a) and the at least one lignin of step b), preferably in an amount of 0.001 to 0.1 wt.-% and most preferably in an amount of 0.005 to 0.05 wt.-%.

According to another embodiment of the present invention, wherein the plants and parts thereof are fruits, vegetables, trees, seeds leaves, wood, nuts, crops, crop plants and flowers.

According to another embodiment of the present invention, the at least one lignin is present in the form of a coating on the at least one calcium carbonate and/or wherein the at least one lignin is a water-insoluble lignin, preferably kraft lignin.

According to another embodiment of the present invention, the cosmetic formulation is a sunscreen product, facial makeup product, hair care product, hair styling product, nail care product, hand care product, skin care product and mixtures thereof.

It should be understood that for the purposes of the present invention, the following terms have the following meanings:

“Chemical sun protection” in the meaning of the present invention, refers to the protection of UV-B and/or UV-A radiation by absorbing the UV-B and/or UV-A radiation.

“Physical sun protection” in the meaning of the present invention, refers to the protection of UV-B and/or UV-A radiation by reflecting the UV-B and/or UV-A radiation.

“UV-A protection” and “UV-B protection” in the meaning of the present invention, refers to the protection from ultraviolet B (UV-B) radiation which ranges from 280 to 320 nm and the ultraviolet A (UV-A) radiation which ranges from >320 to 400 nm, both cause sunburn to plants and parts thereof as well as to human skin.

“Water-insoluble” materials are defined as materials which, when 100 g of said material is mixed with 100 g deionized water and filtered on a filter having a 0.2 μm pore size at 20° C. under atmospheric pressure to recover the liquid filtrate, provide less than or equal to 0.1 g of recovered solid material following evaporation at 95 to 100° C. of 100 g of said liquid filtrate at ambient pressure. “Water-soluble” materials are thus defined as materials which, when 100 g of said material is mixed with 100 g deionized water and filtered on a filter having a 0.2 μm pore size at 20° C. under atmospheric pressure to recover the liquid filtrate, provide more than 0.1 g of recovered solid material following evaporation at 95 to 100° C. of 100 g of said liquid filtrate at ambient pressure.

The “particle size” of particulate materials, for example the calcium carbonate herein is described by its distribution of particle sizes dx. Therein, the value dx represents the diameter relative to which x % by weight of the particles have diameters less than dx. This means that, for example, the d20 value is the particle size at which 20 wt.-% of all particles are smaller than that particle size. The d50 value is thus the weight median particle size, i.e. 50 wt.-% of all grains are bigger and the remaining 50 wt.-% are smaller than this particle size. For the purpose of the present invention the particle size is specified as weight median particle size d50 unless indicated otherwise. The d98 value is the particle size at which 98 wt.-% of all particles are smaller than that particle size. The d98 value is also designated as “top cut”. Particle sizes were determined by using a Sedigraph™ 5100 instrument of Micromeritics Instrument Corporation. The method and the instrument are known to the skilled person and are commonly used to determine the particle size of fillers and pigments. The measurements were carried out in an aqueous solution of 0.1 wt.-% Na4P2O7. The samples were dispersed using a high speed stirrer and sonicated.

A “specific surface area (SSA)” of a calcium carbonate-containing filler material in the meaning of the present invention is defined as the surface area of the calcium carbonate-containing filler material divided by its mass. As used herein, the specific surface area is measured by nitrogen gas adsorption using the BET isotherm (ISO 9277:2010) and is specified in m2/g.

The term “dry” material or “dry” composition, is understood to be a material/composition having less than 5.0% by weight of water relative to the material/composition weight. The % water (equal to residual total moisture content) is determined according to the Coulometric Karl Fischer measurement method, wherein the material/composition is heated to 220° C., and the water content released as vapour and isolated using a stream of nitrogen gas (at 100 ml/min) is determined in a Coulometric Karl Fischer unit.

A “coating” in the meaning of the present invention means that one compound is located on the surface of another compound.

Where the term “comprising” or “containing” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is also to be understood to disclose a group, which preferably consists only of these embodiments.

Where an indefinite or definite article is used when referring to a singular noun, e.g. “a”, “an” or “the”, this includes a plural of that noun unless something else is specifically stated.

Terms like “obtainable” or “definable” and “obtained” or “defined” are used interchangeably. This e.g. means that, unless the context clearly dictates otherwise, the term “obtained” does not mean to indicate that, e.g. an embodiment must be obtained by e.g. the sequence of steps following the term “obtained” even though such a limited understanding is always included by the terms “obtained” or “defined” as a preferred embodiment.

In the following, the details and preferred embodiments of the present inventive will be described in more detail. Embodiments that refer to the compositions and emulsion will also refer to the use of the inventive compositions for sun protection of plants and parts thereof and the use of the inventive emulsion for chemical and physical sun protection in a cosmetic formulation and vice versa.

According to the present invention a dry composition for chemical and physical sun protection is provided, the composition comprising a) at least one calcium carbonate, and b) from 0.1 wt.-% to 100 wt.-%, based on the dry weight of the at least one calcium carbonate of step a) of at least one lignin.

### The at Least One Calcium Carbonate

According to the present invention at least one calcium carbonate is present in the dry composition.

The term “at least one” calcium carbonate in the meaning of the present invention means that the calcium carbonate comprises, preferably consists of, one or more calcium carbonate(s).

In one embodiment of the present invention, the at least one calcium carbonate in the composition comprises, preferably consists of, one calcium carbonate. Alternatively, the at least one calcium carbonate comprises, preferably consists of, two or more calcium carbonates. For example, the at least one calcium carbonate comprises, preferably consists of, two or three calcium carbonates.

The amount of calcium in the calcium carbonate is at least 50 mol.-%, based on the total amount of the calcium carbonate, preferably at least 70 mol.-%, more preferably at least 90 mol.-% and most preferably at least 99 mol.-%. The calcium carbonate may comprise further earth alkali compounds such as Mg.

According to a preferred embodiment of the present invention the calcium carbonate is selected from the group consisting of ground calcium carbonate (GCC), preferably marble, limestone, and/or chalk, precipitated calcium carbonate (PCC), preferably vaterite, calcite and/or aragonite and mixtures thereof.

Natural or ground calcium carbonate (GCC) is understood to be manufactured from a naturally occurring form of calcium carbonate, mined from sedimentary rocks such as limestone or chalk, or from metamorphic marble rocks, eggshells or seashells. Calcium carbonate is known to exist as three types of crystal polymorphs: calcite, aragonite and vaterite. Calcite, the most common crystal polymorph, is considered to be the most stable crystal form of calcium carbonate. Less common is aragonite, which has a discrete or clustered needle orthorhombic crystal structure. Vaterite is the rarest calcium carbonate polymorph and is generally unstable. Ground calcium carbonate is almost exclusively of the calcitic polymorph, which is said to be trigonal-rhombohedral and represents the most stable form of the calcium carbonate polymorphs. The term “source” of the calcium carbonate in the meaning of the present application refers to the naturally occurring mineral material from which the calcium carbonate is obtained. The source of the calcium carbonate may comprise further naturally occurring components such as alumino silicate etc.

In general, the grinding of natural ground calcium carbonate may be a dry or wet grinding step and may be carried out with any conventional grinding device, for example, under conditions such that comminution predominantly results from impacts with a secondary body, i.e. in one or more of: a ball mill, a rod mill, a vibrating mill, a roll crusher, a centrifugal impact mill, a vertical bead mill, an attrition mill, a pin mill, a hammer mill, a pulveriser, a shredder, a de-clumper, a knife cutter, or other such equipment known to the skilled man. In case the calcium carbonate-comprising mineral material comprises a wet ground calcium carbonate-comprising mineral material, the grinding step may be performed under conditions such that autogenous grinding takes place and/or by horizontal ball milling, and/or other such processes known to the skilled man. The wet processed ground calcium carbonate-comprising mineral material thus obtained may be washed and dewatered by well-known processes, e.g. by flocculation, filtration or forced evaporation prior to drying. The subsequent step of drying (if necessary) may be carried out in a single step such as spray drying, or in at least two steps. It is also common that such a mineral material undergoes a beneficiation step (such as a flotation, bleaching or magnetic separation step) to remove impurities.

According to one embodiment of the present invention the source of natural or ground calcium carbonate (GCC) is selected from marble, chalk, limestone, or mixtures thereof. Preferably, the source of ground calcium carbonate is marble. According to one embodiment of the present invention the GCC is obtained by dry grinding. According to another embodiment of the present invention the GCC is obtained by wet grinding and optionally subsequent drying.

According to one embodiment of the present invention, the calcium carbonate comprises one type of ground calcium carbonate. According to another embodiment of the present invention, the calcium carbonate comprises a mixture of two or more types of ground calcium carbonates selected from different sources.

“Precipitated calcium carbonate” (PCC) in the meaning of the present invention is a synthesized material, generally obtained by precipitation following reaction of carbon dioxide and lime in an aqueous environment or by precipitation of a calcium and carbonate ion source in water or by precipitation by combining calcium and carbonate ions, for example CaCl2 and Na2CO3, out of solution. Further possible ways of producing PCC are the lime soda process, or the Solvay process in which PCC is a by-product of ammonia production. Precipitated calcium carbonate exists in three primary crystalline forms: calcite, aragonite and vaterite, and there are many different polymorphs (crystal habits) for each of these crystalline forms. Calcite has a trigonal structure with typical crystal habits such as scalenohedral (S-PCC), rhombohedral (R-PCC), hexagonal prismatic, pinacoidal, colloidal (C-PCC), cubic, and prismatic (P-PCC). Aragonite is an orthorhombic structure with typical crystal habits of twinned hexagonal prismatic crystals, as well as a diverse assortment of thin elongated prismatic, curved bladed, steep pyramidal, chisel shaped crystals, branching tree, and coral or worm-like form. Vaterite belongs to the hexagonal crystal system. The obtained PCC slurry can be mechanically dewatered and dried.

According to one embodiment of the present invention, the precipitated calcium carbonate is precipitated calcium carbonate, preferably comprising aragonitic, vateritic or calcitic mineralogical crystal forms or mixtures thereof.

According to one embodiment of the present invention, the calcium carbonate comprises one type of precipitated calcium carbonate. According to another embodiment of the present invention, the calcium carbonate comprises a mixture of two or more precipitated calcium carbonates selected from different crystalline forms and different polymorphs of precipitated calcium carbonate. For example, the at least one precipitated calcium carbonate may comprise one PCC selected from S-PCC and one PCC selected from R-PCC.

According to a preferred embodiment of the present invention the at least one calcium carbonate-comprising material is ground calcium carbonate, preferably dry ground calcium carbonate. According to another preferred embodiment, the at least one calcium carbonate-comprising material is marble.

It is preferred that the at least one calcium carbonate is a dry ground material, a material being wet ground and dried or a mixture of the foregoing materials. In general, the grinding step can be carried out with any conventional grinding device, for example, under conditions such that refinement predominantly results from impacts with a secondary body, i.e. in one or more of: a ball mill, a rod mill, a vibrating mill, a roll crusher, a centrifugal impact mill, a vertical bead mill an attrition mill, a pin mill, a hammer mill, a pulveriser, a shredder, a de-clumper, a knife cutter, or other such equipment known to the skilled man.

In case the at least one calcium carbonate is a wet ground calcium carbonate, the grinding step may be performed under conditions such that autogenous grinding takes place and/or by horizontal ball milling, and/or other such processes known to the skilled man. The wet processed ground calcium carbonate thus obtained may be washed and dewatered by well-known processes, e.g. by flocculation, filtration or forced evaporation prior to drying. The subsequent step of drying may be carried out in a single step such as spray drying, or in at least two steps, e.g. by applying a first heating step to the calcium carbonate in order to reduce the associated moisture content to a level which is not greater than 5 wt.-%, based on the total dry weight of the calcium carbonate. The residual total moisture content of the filler can be measured by the Karl Fischer coulometric titration method, desorbing the moisture in an oven at 195° C. and passing it continuously into the KF coulometer (Mettler Toledo coulometric KF Titrator C30, combined with Mettler oven DO 0337) using dry N2 at 100 ml/min for 10 min. The residual total moisture content can be determined with a calibration curve and also a blind of 10 min gas flow without a sample can be taken into account. The residual total moisture content may be further reduced by applying a second heating step to the calcium carbonate. In case said drying is carried out by more than one drying steps, the first step may be carried out by heating in a hot current of air, while the second and further drying steps are preferably carried out by an indirect heating in which the atmosphere in the corresponding vessel comprises a surface treatment agent. It is also common that the calcium carbonate is subjected to a beneficiation step (such as a flotation, bleaching or magnetic separation step) to remove impurities.

In one embodiment of the present invention, the at least one calcium carbonate comprises a dry ground calcium carbonate. In another preferred embodiment, the at least one calcium carbonate is a material being wet ground in a horizontal ball mill, and subsequently dried by using the well-known process of spray drying.

Depending on the at least one calcium carbonate, the at least one calcium carbonate according to the present invention has a residual total moisture content of less than 5 wt.-%, preferably less than 4 wt.-%, more preferably less than 3 wt.-%, even more preferably less than 2 wt.-% and most preferably less than 1 wt.-%, based on the total dry weight of the at least one calcium carbonate.

Alternatively, the at least one calcium carbonate according to one embodiment may have a residual total moisture content of from 0.01 to 1 wt.-%, preferably from 0.02 to 0.5 wt.-%, more preferably from 0.03 to 0.3 wt.-%, even more preferably from 0.04 to 0.2 wt.-% and most preferably from 0.05 to 0.15 wt.-%, based on the total dry weight of the at least one calcium carbonate.

For example, in case a wet ground and dried calcium carbonate is used as the at least one calcium carbonate, the residual total moisture content of the at least one calcium carbonate is preferably of from 0.01 to 1 wt.-%, more preferably from 0.02 to 0.1 wt.-% and most preferably from 0.04 to 0.08 wt.-% based on the total dry weight of the at least one calcium carbonate. If a PCC is used as the at least one calcium carbonate, the residual total moisture content of the at least one calcium carbonate is preferably of from 0.01 to 1 wt.-%, more preferably from 0.05 to 0.2 wt.-% and most preferably from 0.05 to 0.15 wt.-%, based on the total dry weight of the at least one calcium carbonate.

According to one embodiment of the present invention, the at least one calcium carbonate is preferably in the form of a particulate material, and may have a particle size distribution as conventionally employed for the material(s) involved in the type of product to be produced. In general, it is preferred that the at least one calcium carbonate has a weight median particle size d50 value in the range from 0.05 μm to 20 μm, preferably from 0.25 μm to 10 μm and most preferably from 0.5 μm to 8 μm.

Additionally or alternatively, the at least one calcium carbonate has a top cut (d98) of ≤100 μm, preferably ≤60 μm, more preferably of ≤45 μm and most preferably of ≤20 μm.

Additionally or alternatively the at least one calcium carbonate has a BET specific surface area of from 0.5 and 100 m2/g, preferably from 0.5 to 50 m2/g, more preferably of from 0.5 to 35 m2/g and most preferably of from 0.5 to 10 m2/g as measured by the BET nitrogen method.

According to another embodiment of the present invention the at least one calcium carbonate has
a) a weight median particle size d₅₀ value in the range from 0.05 μm to 20 μm, preferably from 0.25 μm to 10 μm and most preferably from 0.5 μm to 8 μm, or
b) a top cut (d₉₈) of ≤100 μm, preferably ≤60 μm, more preferably ≤45 μm and most preferably ≤20 μm, or
c) a specific surface area (BET) of from 0.5 to 100 m²/g, preferably from 0.5 to 50 m²/g, more preferably from 0.5 to 35 m²/g and most preferably from 0.5 to 10 m²/g as measured by the BET nitrogen method.

According to another embodiment of the present invention the at least one calcium carbonate has
a) a weight median particle size d₅₀ value in the range from 0.05 μm to 20 μm, preferably from 0.25 μm to 10 μm and most preferably from 0.5 μm to 8 μm, and
b) a top cut (d₉₈) of ≤100 μm, preferably ≤60 μm, more preferably ≤45 μm and most preferably ≤20 μm, and
c) a specific surface area (BET) of from 0.5 to 100 m²/g, preferably from 0.5 to 50 m²/g, more preferably from 0.5 to 35 m²/g and most preferably from 0.5 to 10 m²/g as measured by the BET nitrogen method.

### The at Least One Lignin

According to the present invention at least one lignin is present in the composition in an amount from 0.1 wt.-% to 100 wt.-%, based on the dry weight of the at least one calcium carbonate.

The term “at least one” lignin in the meaning of the present invention means that the lignin comprises, preferably consists of, one or more lignin(s).

In one embodiment of the present invention, the at least one lignin in the composition comprises, preferably consists of, one lignin. Alternatively, the at least one lignin comprises, preferably consists of, two or more lignins. For example, the at least one lignin comprises, preferably consists of, two or three lignins.

Preferably, the at least one lignin in the composition comprises, more preferably consists of, one lignin.

A “lignin” in the meaning of the present invention is defined as an organic biopolymer which is obtained from woods and plants. It is a cross-linked polymer with molecular masses in excess of 10000 u. Lignin is hydrophobic and rich in aromatic subunits and mainly comprises a crosslinked network 4-hydroxy-3-methoxyphenylpropane, 3,5-dimethoxy-4-hydroxyphenylpropane, and 4-hydroxyphenylpropane.

Lignin is known to the skilled person and commercially available, for example from Domsjö under the trade name Domsjö Lignin DS10.

Plant lignins can be broadly divided into three classes: softwood (gymnosperm), hardwood (angiosperm) and grass or annual plant (graminaceous) lignin. Three different phenylpropane units, or monolignols, are responsible for lignin biosynthesis. Guaiacyl lignin is composed principally of coniferyl alcohol units, while guaiacyl-syringyl lignin contains monomeric units from coniferyl and sinapyl alcohol. In general, guaiacyl lignin is found in softwoods while guaiacyl-syringyl lignin is present in hardwoods. Graminaceous lignin is composed mainly of p-coumaryl alcohol units. Lignin polymerization is initiated by oxidation of the phenylpropane phenolic hydroxyl groups. Stabilization of the radical occurs by coupling to another radical in any of the positions of the unpaired electron.

According to one embodiment of the present invention, the at least one lignin is a water soluble or water insoluble lignin selected from the group consisting of natural lignin, klason lignin, hydrolyzed lignin, milled wood lignin, soda lignin, organosolv lignin, kraft lignin, sulphonated lignin and mixtures thereof.

Natural lignin is the lignin that is present in plant tissues and is also known as native lignin.

Klason lignin is the acid insoluble lignin content in natural lignin. It is obtained by pre-hydrolysis of natural lignin in H2SO4, hydrolyzing the mixture at high temperatures and filtration. The retentate comprises the klason lignin. The Klason process is known to the skilled person.

Hydrolyzed lignin is obtained by refluxing lignin or lignocellulose with HCl in a dioxane/water composition. The treatment results in the degradation of lignin with formation of substantial amounts of arylpropanes and the majority of the acidolysis monomers originate from arylglycerol β-aryl ether structure.

Milled wood lignin (MWL) also known as Bjorkman lignin is obtained by grinding wood meal in a ball mill either dry or in the presence of nonswelling solvents such as, for example, toluene, wherein the cell structure of the wood is destroyed. A portion of lignin can be obtained from the suspension by extraction with a dioxane-water mixture. The Bjorkman process is known to the skilled person.

Kraft lignin is the lignin obtained from the Kraft process also known as kraft pulping or sulfate process. The process is for the conversion of wood into wood pulp, which consists of almost pure cellulose fibers, the main component of paper and is known to the skilled person. The Kraft process entails treatment of wood chips with a hot mixture of water, sodium hydroxide (NaOH), and sodium sulfide (Na2S), known as white liquor, that breaks the bonds that link lignin, hemicellulose, and cellulose. The technology entails several steps, both mechanical and chemical.

Lignosulfonate also known as sulphonated lignin are water-soluble anionic polyelectrolyte polymers. They are obtained from wood by treating wood at elevated temperatures with solutions containing sulfur dioxide and hydrogen sulfite ions. This process is also known to the skilled person.

Soda lignin is obtained from the soda process which involves heating fibrous wood material in a pressurized reactor at high temperature in the presence of sodium hydroxide (i.e. soda), also known as cooking liquor. In the process, lignin is separating from the cellulose, and is suspended in the liquid phase, which is called black liquor. The black liquor therefore contains lignin and sodium hydroxide (soda) and is known as soda lignin.

Organosolv lignin is obtained by the organosolv process. The organosolv process includes extracting lignin from lignocellulosic biomass using organic solvents typically with an acidic catalyst. Organosolv lignin does not comprise sulfur or sulfonate groups and has a molecular weight of about 1000 to 2000 g/mole.

According to one embodiment of the present invention the at least one lignin is a mixture of two or more lignins e.g. is a mixture of two lignins, for example, sulphonated lignin and a further lignin, for example, kraft lignin or natural lignin.

According to a preferred embodiment of the present invention, the at least one lignin consist only of one lignin, preferably selected from natural lignin, klason lignin, hydrolyzed lignin, milled wood lignin, soda lignin, organosolv lignin, kraft lignin, or sulphonated lignin and most preferably consist merely of kraft lignin.

According to one embodiment of the present invention the at least one lignin is water soluble lignin. According to another embodiment of the present invention the at least one lignin is water insoluble lignin. Preferably, the at least one lignin is water insoluble lignin and most preferably the at least one water insoluble lignin is selected from the group consisting of klason lignin, kraft lignin and mixtures thereof and most preferably is kraft lignin.

According to another embodiment of the present invention the at least one lignin has a molecular mass above 10000 u, preferably between 15000 and 1000000 u even more preferably between 50000 to 800000 u and most preferably between 100000 and 500000 u.

The at least one lignin is present in the composition in an amount from 0.1 to 100 wt.-% based on the dry weight of the at least one calcium carbonate of step a). According to a preferred embodiment of the present invention, the at least one lignin is present in the composition in an amount from 1 to 50 wt.-% based on the dry weight of the at least one calcium carbonate of step a), preferably in an amount of 3 to 30 wt.-%, and most preferably in an amount of 5 to 25 wt.-%.

### The Dry Composition

The inventive dry composition for chemical and physical sun protection comprises a) at least one calcium carbonate, and b) from 0.1 wt.-% to 100 wt.-%, based on the dry weight of the at least one calcium carbonate of step a) of at least one lignin.

Alternatively the inventive dry composition for chemical and physical sun protection consists of a) at least one calcium carbonate, and b) from 0.1 wt.-% to 100 wt.-%, based on the dry weight of the at least one calcium carbonate of step a) of at least one lignin.

Preferably, the inventive dry composition for chemical and physical sun protection comprises a) at least one calcium carbonate, and b) from 0.1 wt.-% to 100 wt.-%, based on the dry weight of the at least one calcium carbonate of step a) of at least one lignin. In that case other compounds may be present in the composition.

According to one embodiment of the present invention, the composition further comprises an organic solvent.

An “organic solvent” in the meaning of the present invention is a compound that is liquid at room temperature and that is different from water. Room temperature refers to a temperature of 25° C. Preferably the organic solvent is able to dissolve at least one lignin and preferably a non-water soluble lignin.

The organic solvent of the present invention can be any organic solvent that is suitable for the inventive composition. The skilled person knows how to select such an organic solvent. Organic solvents are known to the skilled person and are commercially available.

According to one embodiment of the present invention, the organic solvent is selected from the group consisting of hexane, toluene, methanol, ethanol, dioxane, acetone, dimethyl sulfoxide, dimethylformamide, ethylene glycol, ethylacetate, glycerol, γ-valerolactone, polyethylene glycol, polypropylene glycol and mixtures thereof, more preferably is selected from the group consisting of ethylene glycol, ethylacetate, glycerol, γ-valerolactone, polyethylene glycol, polypropylene glycol and mixtures thereof, and most preferably is γ-valerolactone.

Hexane is an alkane of six carbon atoms, with the chemical formula C6H14 and comprises five structural isomers. All hexanes are colorless liquids, odorless when pure, with boiling points between 50 and 70° C.

Toluene is an aromatic hydrocarbon with the formula C6H5CH3 and is a colorless, water-insoluble liquid with the smell associated with paint thinners.

Methanol, also known as methyl alcohol among others, is a chemical with the formula CH3OH. Ethanol, also known as ethyl alcohol among others, is a chemical with the formula CH3-CH2-OH.

Dioxane is a heterocyclic organic compound, classified as an ether. It is a colorless liquid with a faint sweet odor. Dioxane comprises the isomers 1,2-dioxane, 1,3-dioxane and 1,4-dioxane.

Acetone, or propanone, is the organic compound with the formula (CH3)2CO. It is a colorless, volatile, flammable liquid and is the simplest and smallest ketone.

Dimethyl sulfoxide (DMSO) is an organosulfur compound with the formula (CH3)2SO. This colorless liquid is a polar aprotic solvent that dissolves both polar and nonpolar compounds and is miscible in a wide range of organic solvents as well as water.

Dimethylformamide is an organic compound with the formula (CH3)2NC(O)H. Commonly abbreviated as DMF, this colorless liquid is miscible with water and the majority of organic liquids.

Ethylene glycol also known as ethane-1,2-diol is an organic compound with the chemical formula (CH2OH)2. It is an odorless, colorless, sweet-tasting, viscous liquid.

Ethyl acetate is an organic compound with the formula CH3-COO—CH2-CH3. It is a colorless liquid and has a characteristic sweet smell. Ethyl acetate is the ester of ethanol and acetic acid.

Glycerol is also called glycerine or glycerin or propane-1,2,3-triol and is a simple polyol compound. It is a colorless, odorless, viscous liquid that is sweet-tasting and non-toxic. It has the chemical formula CH2OH—CHOH—CH2OH.

γ-valerolactone is an organic compound with the formula C5H8O2. This colorless liquid is chiral but is usually used as the racemate. It is readily obtained from cellulosic biomass and is a potential fuel and green solvent.

Polyethylene glycol is a polyether compound with many applications, from industrial manufacturing to medicine. PEG is also known as polyethylene oxide (PEO) or polyoxyethylene (POE), depending on its molecular weight. The structure of PEG is commonly expressed as H—(O—CH2-CH2)n-OH.

Polypropylene glycol or polypropylene oxide is the polymer of propylene glycol. Chemically it is a polyether. The structure of PPG is commonly expressed as H—(O—CHCH3-CH2)n-OH.

According to a preferred embodiment of the present invention the solvent is γ-valerolactone.

According to one embodiment of the present invention the organic solvent is present in the composition in an amount of 5 to 50 wt.-%, based on the dry weight of the at least one calcium carbonate of step a), more preferably in an amount of 10 to 40 wt.-% and most preferably in an amount of 15 to 35 wt.-%.

Additionally or alternatively, the organic solvent is present in the composition in an amount of 100 to 500 wt.-%, based on the dry weight of the at least one lignin of step b), more preferably in an amount of 150 to 450 wt.-% and most preferably in an amount of 200 to 300 wt.-%.

According to one embodiment of the present invention the organic solvent is present in the composition in an amount of 5 to 50 wt.-%, based on the dry weight of the at least one calcium carbonate of step a), more preferably in an amount of 10 to 40 wt.-% and most preferably in an amount of 15 to 35 wt.-% or in an amount of 100 to 500 wt.-%, based on the dry weight of the at least one lignin of step b), more preferably in an amount of 150 to 450 wt.-% and most preferably in an amount of 200 to 300 wt.-%.

According to another embodiment of the present invention the organic solvent is present in the composition in an amount of 5 to 50 wt.-%, based on the dry weight of the at least one calcium carbonate of step a), more preferably in an amount of 10 to 40 wt.-% and most preferably in an amount of 15 to 35 wt.-% and in an amount of 100 to 500 wt.-%, based on the dry weight of the at least one lignin of step b), more preferably in an amount of 150 to 450 wt.-% and most preferably in an amount of 200 to 300 wt.-%.

According to one embodiment of the present invention the dry composition comprises a) at least one calcium carbonate, b) from 0.1 wt.-% to 100 wt.-%, based on the dry weight of the at least one calcium carbonate of step a) of at least one lignin and c) an organic solvent.

According to another embodiment of the present invention the dry composition consist of a) at least one calcium carbonate, b) from 0.1 wt.-% to 100 wt.-%, based on the dry weight of the at least one calcium carbonate of step a) of at least one lignin and c) an organic solvent.

The organic solvent may further comprise water or may be used in combination with water. However, the amount of water in the organic solvent or when used in combination with the organic solvent is rather low. More precisely, even if water is present in the organic solvent or is used in combination with the organic solvent still a dry composition according to the present invention is obtained.

As already set out above, a “dry” composition, is understood to be a composition having less than 5.0% by weight of water relative to the composition weight. Preferably, the dry composition according to the present invention comprises less than 4 wt.-%, more preferably less than 3 wt.-%, even more preferably less than 2 wt.-% and most preferably less than 1 wt.-% water, based on the total dry weight of the composition.

According to one embodiment of the present invention, the at least one lignin is present in the composition in form of a mixture with the at least one calcium carbonate. In that case the at least one lignin and the at least one calcium carbonate coexist in the inventive composition.

According to another embodiment of the present invention, the at least one lignin is present in the composition in the form of a coating on the at least one calcium carbonate. In that case the at least one lignin is located on the surface of the at least one calcium carbonate.

According to a preferred embodiment of the present invention the at least one lignin is present in the composition in the form of a coating on the at least one calcium carbonate. Preferably, the at least one lignin in the coating on the at least one calcium carbonate is a water insoluble lignin, and preferably kraft lignin.

The skilled person knows how to prepare coated particles. Preferably these coated particles are prepared by mixing the components of the inventive composition in the presence of an organic solvent and afterwards drying the mixture.

Mixing may be done consecutively in any order or simultaneously. For example, the at least one lignin may be solved in the organic solvent and afterwards, the at least one calcium carbonate may be added. Alternatively, the at least one calcium carbonate may be dispersed in the organic solvent and afterwards, the at least one lignin may be added. According to another embodiment all the compounds may be mixed simultaneously.

The mixing may be carried out under conventional mixing conditions. The skilled person will adapt these mixing conditions (such as the configuration of mixing pallets and mixing speed) according to his process equipment. It is appreciated that any mixing method which would be suitable may be used.

In one embodiment, mixing is carried out at a temperature in the range from 15 to 120° C., more preferably from 20 to 110° C. and most preferably from 30 to 100° C. Mixing can be performed for at least 1 s, at least 10 s, at least 30 s, at least 1 min, at least 10 min or at least 1 h.

The drying may be carried out in a single step such as spray drying, or in at least two steps, e.g. by applying a first heating step to the calcium carbonate in order to reduce the associated moisture content. The residual total moisture content may be further reduced by applying a second heating step to the calcium carbonate. In case said drying is carried out by more than one drying steps, the first step may be carried out by heating in a hot current of air, while the second and further drying steps are preferably carried out by an indirect heating.

The obtained at least one calcium carbonate that is coated with the at least one lignin may be further deagglomerated, for example, during a grinding step. In general, the grinding step can be carried out with any conventional grinding device, for example, under conditions such that refinement predominantly results from impacts with a secondary body, i.e. in one or more of: a ball mill, a rod mill, a vibrating mill, a roll crusher, a centrifugal impact mill, a vertical bead mill an attrition mill, a pin mill, a hammer mill, a pulveriser, a shredder, a de-clumper, a knife cutter, or other such equipment known to the skilled man.

According to one embodiment of the present invention, the composition of the present invention is in solid form, preferably in form of a particulate material. The term “particulate” in the meaning of the present application refers to materials composed of a plurality of particles. Said plurality of particles may be defined, for example, by its particle size distribution. The expression “particulate material” may comprise powders, grains, tablets, flakes or crumbles.

According to another embodiment of the present invention, the composition of the present invention is in form of a slurry. A “suspension” or “slurry” in the meaning of the present invention comprises undissolved solids and a solvent as defined above, and optionally further additives, and usually contains large amounts of solids and, thus, is more viscous and can be of higher density than the liquid from which it is formed.

For example, the amount of the at least one calcium carbonate and the at least one lignin is from 0.1 to 20 wt.-%, based on the total weight of the slurry, preferably from 0.2 to 15 wt.-%, more preferably in an amount of 0.5 to 10 wt.-% and most preferably in an amount of 1 to 5 wt.-%.

According to a preferred embodiment of the present invention the dry formulation contains at least one calcium carbonate, preferably ground calcium carbonate and 10 wt.-% of at least one lignin, preferably alkali lignin, based on the dry weight of the at least one calcium carbonate. Preferably the lignin is present as a coating on the at least one calcium carbonate. According to another preferred embodiment the dry composition further comprises an organic solvent, preferably γ-valerolactone, in an amount of 30 wt.-% based on the dry weight of the at least one calcium carbonate and in an amount of 300 wt.-% based on the dry weight of the lignin. According to a preferred embodiment of the present invention, the dry formulation comprises, preferably consists of ground calcium carbonate and 10 wt % of alkali lignin, based on the dry weight of the at least one calcium carbonate, as well as γ-valerolactone, in an amount of 30 wt.-% based on the dry weight of the at least one calcium carbonate and in an amount of 300 wt.-% based on the dry weight of the lignin.

### The Fluid Composition

The fluid composition for chemical and physical sun protection of the present invention comprises water and 1 to 100 wt.-% of the dry composition according to the present invention, based on the weight of the water.

The dry composition has already been described in detail above.

The water of the present invention may be selected from drinking water, process water, demineralized water, distilled water, rain water, recycled water, river water and mixtures thereof. According to a preferred embodiment of the present invention the water present in the fluid composition is drinking water.

Drinking water, also known as potable water, is water that is safe to drink or to use for food preparations. Rain water/river water is obtained from rain/rivers. Recycled water is water that has been recycled and can be used in agriculture. Process water is water which is not considered drinkable and is basically used in relation to industrial plants, industrial processes and production facilities. Demineralized water is specially purified water that has had most or all of its mineral and salt ions removed, such as calcium, magnesium, sodium, chloride, sulphate, nitrate and bicarbonate. It is also known as deionized water. Distilled water is water that has been boiled into vapor and condensed back into liquid in a separate container.

According to one embodiment of the present invention the fluid composition comprises water and 1 to 99.8 wt.-% of the dry composition according to the present invention, based on the weight of the water, preferably 5 to 95 wt.-%, even more preferably 10 to 90 wt.-% and most preferably 15 to 85 wt.-%.

According to one embodiment of the present invention, the fluid composition further comprises a surfactant. A “surfactant” in the meaning of the present invention is an agent that lowers the surface tension (or interfacial tension) between two different compounds.

Any surfactant can be present in the inventive composition that is suitable for use in combination with calcium carbonate and/or lignin. Such surfactants are known to the skilled person and are commercially available.

According to a preferred embodiment of the present invention the surfactant is selected from the group consisting of monomers and/or co-monomers of acrylic acid, methacrylic acid, itaconic acid, crotonic acid, fumaric acid, maleic anhydride acid, isocrotonic acid, aconitic acid (cis or trans), mesaconic acid, sinapinic acid, undecylenic acid, angelic acid, canellic acid, hydroxyacrylic acid, acrolein, acrylamide, acrylonitrile, dimethylaminoethyl methacrylate, vinylpyrrolidone, vinylcaprolactam, ethylene, propylene, isobutylene, diisobutylene, vinyl acetate, styrene, α-methyl styrene, methyl vinyl ketone, the esters of acrylic and methacrylic acids, organo modified trisiloxane, PEG, polyglycerol-ester, sophorolipid, polyether and mixtures thereof, and most preferably is poly(acrylic acid) and/or poly (methacrylic acid).

Additionally or alternatively, the surfactant is present in the fluid composition in an amount of 0.0001 to 0.2 wt.-%, based on the dry weight of the at least one calcium carbonate of step a) and the at least one lignin of step b), preferably in an amount of 0.001 to 0.1 wt.-% and most preferably in an amount of 0.005 to 0.05 wt.-%.

According to a preferred embodiment of the present invention the composition comprises a surfactant, preferably selected from the group consisting of monomers and/or co-monomers of acrylic acid, methacrylic acid, itaconic acid, crotonic acid, fumaric acid, maleic anhydride acid, isocrotonic acid, aconitic acid (cis or trans), mesaconic acid, sinapinic acid, undecylenic acid, angelic acid, canellic acid, hydroxyacrylic acid, acrolein, acrylamide, acrylonitrile, dimethylaminoethyl methacrylate, vinylpyrrolidone, vinylcaprolactam, ethylene, propylene, isobutylene, diisobutylene, vinyl acetate, styrene, α-methyl styrene, methyl vinyl ketone, the esters of acrylic and methacrylic acids, organo modified trisiloxane, PEG, polyglycerol-ester, sophorolipid, polyether and mixtures thereof, and most preferably is poly(acrylic acid) and/or poly (methacrylic acid) or,

wherein the surfactant is present in the composition in an amount of 0.0001 to 0.2 wt.-%, based on the dry weight of the at least one calcium carbonate of step a), preferably in an amount of 0.001 to 0.1 wt.-% and most preferably in an amount of 0.005 to 0.05 wt.-%.

According to another preferred embodiment of the present invention, the composition comprises a surfactant, preferably selected from the group consisting of monomers and/or co-monomers of acrylic acid, methacrylic acid, itaconic acid, crotonic acid, fumaric acid, maleic anhydride acid, isocrotonic acid, aconitic acid (cis or trans), mesaconic acid, sinapinic acid, undecylenic acid, angelic acid, canellic acid, hydroxyacrylic acid, acrolein, acrylamide, acrylonitrile, dimethylaminoethyl methacrylate, vinylpyrrolidone, vinylcaprolactam, ethylene, propylene, isobutylene, diisobutylene, vinyl acetate, styrene, α-methyl styrene, methyl vinyl ketone, the esters of acrylic and methacrylic acids, organo modified trisiloxane, PEG, polyglycerol-ester, sophorolipid, polyether and mixtures thereof, and most preferably is poly(acrylic acid) and/or poly (methacrylic acid) and,

wherein the surfactant is present in the composition in an amount of 0.0001 to 0.2 wt.-%, based on the dry weight of the at least one calcium carbonate of step a), preferably in an amount of 0.001 to 0.1 wt.-% and most preferably in an amount of 0.005 to 0.05 wt.-%.

Such surfactants are known to the skilled person and are commercially available. According to an exemplarily embodiment the surfactant is polyether trisiloxane (Break-Thru S 200) and is commercially available from Evonik Nutrition & Care GmbH.

According to a preferred embodiment of the present invention the inventive fluid composition comprises an organic solvent as defined above in addition to water.

Preferably the organic solvent is miscible with water in any ratio and preferably the ratio of water:organic solvent is from 100:0.1 to 100:200, preferably from 100:1 to 100:150, more preferably from 100:5 to 100:120 and most preferably from 100:10 to 100:100, based on the weight of the water and the dry weight of the organic solvent.

According to one embodiment of the present invention, the fluid composition merely consists of calcium carbonate, lignin and water. According to another preferred embodiment of the present invention, the fluid composition merely consists of calcium carbonate, lignin, an organic solvent and water. According to another preferred embodiment of the present invention, the fluid composition merely consists of calcium carbonate, lignin, an organic solvent, a surfactant and water.

According to one embodiment of the present invention the water is present in the fluid composition in an amount of 0.1 to 50 wt.-%, based on the dry weight of the at least one calcium carbonate of step a) and the dry weight of the at least one lignin of step b), more preferably in an amount of 0.5 to 40 wt.-% and most preferably in an amount of 1 to 35 wt.-%.

The skilled person knows how to prepare such fluid compositions. Preferably, these fluid compositions are prepared by mixing the water and the dry composition according to the present invention. Alternatively, these fluid compositions are prepared by mixing the water and the components of the dry composition according to the present invention.

Mixing may be done consecutively in any order or simultaneously. For example, the dry composition may be added to the water in one or several portions. Alternatively, the at least one calcium carbonate, the at least one lignin and the optional organic solvent may be added to the water in any order. According to another embodiment all these compounds may be mixed simultaneously.

The mixing may be carried out under conventional mixing conditions. The skilled person will adapt these mixing conditions (such as the configuration of mixing pallets and mixing speed) according to his process equipment. It is appreciated that any mixing method which would be suitable may be used.

In one embodiment, mixing is carried out at a temperature in the range from 15 to 100° C., more preferably from 20 to 95° C. and most preferably from 30 to 90° C. Mixing can be performed for at least 1 s, at least 10 s, at least 30 s, at least 1 min, at least 10 min or at least 1 h.

The fluid composition of the present invention is in form of a slurry. A “suspension” or “slurry” in the meaning of the present invention comprises undissolved solids and a solvent, and optionally further additives, and usually contains large amounts of solids and, thus, is more viscous and can be of higher density than the liquid from which it is formed.

For example, the amount of the at least one calcium carbonate and the at least one lignin is from 0.1 to 20 wt.-%, based on the total weight of the slurry, preferably from 0.2 to 15 wt.-%, more preferably in an amount of 0.5 to 10 wt.-% and most preferably in an amount of 1 to 5 wt.-%.

According to a preferred embodiment the fluid composition comprises about 5 wt.-% of the dry composition based on the weight of the water and about 0.05 wt. % of surfactant based on the dry weight of the at least one calcium carbonate of step a) and the at least one lignin of step b). According to a preferred embodiment of the present invention, the fluid composition comprises, preferably consists of 5 wt.-% of a dry composition, based on the weight of the water, and about 0.05 wt. % of surfactant based on the dry weight of the at least one calcium carbonate of step a) and the at least one lignin of step b), wherein the dry formulation comprises, preferably consists of ground calcium carbonate and 10 wt.-% of alkali lignin, based on the dry weight of the at least one calcium carbonate, as well as γ-valerolactone, in an amount of 30 wt.-% based on the dry weight of the at least one calcium carbonate and in an amount of 300 wt.-% based on the dry weight of the lignin. Preferably the surfactant is polyether trisiloxane.

### The Emulsion

The emulsion for chemical and physical sun protection of the present invention comprises a water in oil or oil in water mixture and 0.1 to 100 wt.-% of the dry composition according to the present invention, based on the weight of the water in oil or oil in water mixture.

The dry composition has already been described in detail above. Furthermore, the water has also already been described above.

An emulsion in the meaning of the present invention is a mixture of two or more liquids that are normally immiscible and one liquid (the dispersed phase) is dispersed in the other (the continuous phase). An oil-in-water emulsion according to the present invention is an emulsion, wherein the oil is the dispersed phase, and water is the continuous phase. A water-in-oil emulsion according to the present invention is an emulsion, wherein water is the dispersed phase and oil is the continuous phase.

An oil in the meaning of the present invention is a liquid or solid silicon and/or hydrocarbon containing compound. The oil of the present invention can be any oil that is suitable for the inventive composition. The skilled person knows how to select such an oil. Oils are known to the skilled person and are commercially available.

Any oil known to the skilled person as being suitable in cosmetic formulations may be used. For example, the oil may be selected from the group comprising alkanecoconutester, polydimethylsiloxanes, polyalkylmethylsiloxanes, silicones, petroleum jelly such as vaseline, vegetable oils such as palm oil, esters of vegetable oils, and mixtures thereof. Preferably, the at least one oil is alkanecoconutester or vaseline.

Preferably the emulsion according to the present invention comprises water and oil in a ratio of water:oil from 100:0.1 to 100:1000, preferably from 100:1 to 100:700, more preferably from 100:5 to 100:500 and most preferably from 100:10 to 100:300, based on the weight of the water and the dry oil.

The emulsion according to the present invention may comprise further compounds such as dispersing agents, emulsifiers, preservatives, active agents, cosmetic ingredients, colored pigments, skin active substances etc.

According to one embodiment of the present invention, the emulsion merely consists of calcium carbonate, lignin and water in oil or oil in water mixture. According to another preferred embodiment of the present invention, the emulsion merely consists of calcium carbonate, lignin, an organic solvent and water in oil or oil in water mixture. According to another preferred embodiment of the present invention, the emulsion merely consists of calcium carbonate, lignin, an organic solvent, a skin active substance and water in oil or oil in water mixture.

According to one embodiment of the present invention the emulsion comprises water in oil or oil in water mixture and 0.1 to 15 wt.-% of the dry composition according to the present invention, based on the weight of the water in oil or oil in water mixture, preferably 0.25 to 10 wt.-%, even more preferably 0.5 to 5 wt.-% and most preferably 1 to 3 wt.-%.

Alternatively, the amount of the at least one calcium carbonate and the at least one lignin is from 0.1 to 15 wt.-%, based on the total weight of the emulsion, preferably from 0.25 to 10 wt.-%, more preferably in an amount of 0.5 to 5 wt.-% and most preferably in an amount of 1 to 3 wt.-%.

The skilled person knows how to prepare such emulsions. Preferably, these emulsions are prepared by first preparing the water in oil or oil in water mixtures and afterwards mixing the dry composition according to the present invention with these emulsions. Alternatively, these emulsions are prepared by mixing all the components and afterwards emulsifying these mixtures.

Mixing and emulsifying may be done consecutively in any order or simultaneously. For example, the dry composition may be added to the water in oil or oil in water mixture in one or several portions. Alternatively, the at least one calcium carbonate, the at least one lignin and the optional organic solvent may be added to the water in oil or oil in water mixture in any order. According to another embodiment all these compounds may be mixed simultaneously. Alternatively, the dry composition may be mixed with water and oil in any order or simultaneously in one or several portions and, afterwards, this mixture is emulsified.

The mixing and/or emulsifying may be carried out under conventional mixing and/or emulsifying conditions. The skilled person will adapt these mixing and/or emulsifying conditions (such as the configuration of mixing pallets and mixing speed) according to his process equipment. It is appreciated that any mixing and/or emulsifying method which would be suitable may be used.

In one embodiment, mixing and/or emulsifying is carried out at a temperature in the range from 15 to 100° C., more preferably from 20 to 95° C. and most preferably from 30 to 90° C. Mixing and/or emulsifying can be performed for at least 1 s, at least 10 s, at least 30 s, at least 1 min, at least 10 min or at least 1 h.

According to a preferred embodiment of the present invention the emulsion is a water in oil emulsion and comprises 0.25 to 10 wt.-%, preferably about 5 wt.-% of the dry composition according to the present invention, based on the weight of the water in oil mixture. Preferably the emulsion according to the present invention comprises the water and the oil in a ratio of water:oil from 100:10 to 100:300, based on the weight of the water and the dry oil and preferably in a ratio of about 100:200. The dry composition according to the present invention preferably comprises about 10 wt.-% of at least one lignin, based on the dry weight of the at least one calcium carbonate and preferably alkali lignin.

The inventors surprisingly found out that the foregoing compositions namely the dry compositions and the fluid compositions as well as the emulsions provide sufficient or improved chemical and physical sun protection. More precisely, the inventors surprisingly found out that the inventive compositions offer two different mechanisms for UV-B and/or UV-A protection to living cells, especially to plants and parts thereof as well as to human skin by different mechanisms, namely by reflecting the UV-B and/or UV-A radiation as well as by absorbing the UV-B and/or UV-A radiation.

UV-B and/or UV-A protection can be measured by transmittance and absorbance measurements. Ultraviolet-visible spectroscopy or ultraviolet-visible spectrophotometry and Near Infrared spectroscopy (UV-Vis or UV/Vis and NIR) refers to absorption spectroscopy or reflectance spectroscopy in the ultraviolet-visible and near infrared spectral region. This means it uses light in the visible and adjacent ranges. The absorption or reflectance in this range directly affects the perceived color of the chemicals involved. In this region of the electromagnetic spectrum, atoms and molecules undergo electronic transitions. As used herein, the transmittance and absorbance is measured by a double beam PerkinElmer Lambda 950 UV/Vis/NIR spectrometer equipped with a 150 mm integrating sphere with PMT and InGaAs detectors.

According to one embodiment of the present invention the composition/emulsion of the present invention has an improved UV-B and/or UV-A protection to living cells, for example to plants and parts thereof, especially to fruits, as well as to human skin, compared to an identical composition/emulsion that comprises merely at least one calcium carbonate or at least one lignin.

An “identical composition/identical emulsion” in the meaning of the present invention refers to a composition/emulsion that consists of the same ingredients in the same amounts than the inventive composition/emulsion with the exception, that the composition does not comprise both of at least one calcium carbonate and at least one lignin, but only calcium carbonate or only lignin. The missing component is replaced by the other component.

According to one preferred embodiment of the present invention the composition/emulsion of the present invention has an improved transmittance and absorbance in the range of 280 nm to 320 nm, compared to an identical composition/emulsion that comprises merely at least one calcium carbonate, or alternatively in the range of >320 nm to 400 nm, and most preferably in the range of 280 nm to 400 nm.

Furthermore, the inventors surprisingly found that the compositions/emulsion of the present invention can be easily and quickly produced, are cheap and especially easy to handle. More precisely, the compositions/emulsion of the present invention can be easily produced by mixing and/or emulsifying the components of the inventive composition/emulsion as described above.

Furthermore, in case the at least one lignin is present in the composition/emulsion in the form of a coating on the at least one calcium carbonate the inventors found out that these coated particles offer two different mechanisms for UV-B and/or UV-A protection to living cells, especially to plants and parts thereof as well as to human skin by different mechanisms, namely by reflecting the UV-B and/or UV-A radiation as well as by absorbing the UV-B and/or UV-A radiation. Since calcium carbonate and the lignin are non-toxic to humans and do not provide a harmful effect on the environment also these coated particles are non-toxic to humans and do not provide a harmful effect on the environment. Furthermore, in case the at least one lignin in the coating on the at least one calcium carbonate is a water insoluble lignin, for example kraft lignin, the inventors surprisingly found out that the coating sticks to the calcium carbonate even after redispersing the coated calcium carbonate particles in water. Therefore, the coating layer is preserved even if the particles are dispersed in water. For example, if such a composition is located on plants and parts thereof, the lignin cannot be washed easily away by rain or by irrigation water. If such a composition is incorporated in a cosmetic formulation on water basis, the lignin will not be washed away from the human skin by sweat easily since it sticks to the surface of the calcium carbonate.

### Use of the Compositions and the Emulsion

The inventive compositions and the inventive emulsion are used for sun protection.

More precisely, the inventive compositions comprising the inventive dry composition and the inventive fluid composition as used for sun protection of plants and parts thereof, where the sun protection includes physical and chemical protection.

This can be easily done by applying the inventive composition to plants and parts thereof by any suitable method known to the skilled person, for example, by spraying, painting or dipping. According to a preferred method the inventive composition is sprayed onto plants and parts thereof. Equipment for spraying the composition is known to the skilled person and commercially available.

According to one embodiment of the present invention, the plants and parts thereof are fruits, vegetables, trees, seeds, leaves, wood, nuts, crops, crop plants and flowers and especially are fruits like apples, oranges, citrons, cherries, pears, plums, bananas or mango.

As already set out above the inventors surprisingly found out that the foregoing compositions provide sufficient sun protection to plants and parts thereof, especially to fruits. Especially, the inventors surprisingly found that when a composition comprising a) at least one calcium carbonate and b) from 0.1 wt.-% to 100 wt.-%, based on the dry weight of at least one calcium carbonate of step a) of at least one lignin is used, the composition provides improved sun protection, namely sun protection which includes physical as well as chemical protection to plants and parts thereof. Furthermore, when the composition according to the present invention is used for sun protection of plants and parts thereof, another advantage might be that the composition further provides calcium nutrients to the plants due to the calcium carbonate in the composition. Furthermore, another advantage might be that the layer of the composition that forms on the plants and parts thereof, when the fluid composition is used on the plants and parts thereof repels also insects.

Furthermore, the present invention refers to the use of an emulsion according to the present invention. More precisely, the present invention refers to the use of an emulsion comprising a water in oil or oil in water mixture and 1 to 100 wt.-% of the dry composition according to the present invention, based on the weight of the water in oil or oil in water mixture for chemical and physical sun protection in a cosmetic formulation.

This can be easily done by applying the inventive emulsion into a cosmetic formulation by any suitable method known to the skilled person, for example, by mixing, dispersing or emulsifying. According to a preferred method the ingredients of the cosmetic formulation are mixed with the inventive emulsion. Equipment therefore is known to the skilled person and commercially available.

According to one embodiment of the present invention, the cosmetic formulation is a sunscreen product, facial makeup product, hair care product, hair styling product, nail care product, hand care product, skin care product and mixtures thereof.

According to another preferred embodiment the at least one lignin which is present in the cosmetic formulation in the form of a coating on the at least one calcium carbonate. Additionally or alternatively, the at least one lignin in the cosmetic formulation is a water-insoluble lignin and preferably kraft lignin.

According to another preferred embodiment the at least one lignin which is present in the cosmetic formulation in the form of a coating on the at least one calcium carbonate and the at least one lignin in the cosmetic formulation is a water-insoluble lignin and preferably kraft lignin.

According to another preferred embodiment the at least one lignin which is present in the cosmetic formulation in the form of a coating on the at least one calcium carbonate or the at least one lignin in the cosmetic formulation is a water-insoluble lignin and preferably kraft lignin.

As already set out above the inventors surprisingly found out that the foregoing compositions provide sufficient sun protection to living cells and especially to human skin. The inventors surprisingly found that when an emulsion comprising a water in oil or oil in water mixture and 1 to 100 wt.-% of the dry composition according to the present invention, based on the weight of the water in oil or oil in water mixture is used, the composition provides improved sun protection, namely sun protection which includes physical as well as chemical protection to human skin.

Furthermore, the present invention refers to a cosmetic formulation comprising the above mentioned inventive emulsion. More precisely, the present invention refers to a cosmetic formulation comprising an emulsion comprising a water in oil or oil in water mixture and 1 to 100 wt.-% of the dry composition according to the present invention, based on the weight of the water in oil or oil in water mixture.

According to one embodiment the at least one lignin is present in the cosmetic formulation in the form of a coating on the at least one calcium carbonate and/or wherein the at least one lignin is a water-insoluble lignin, preferably kraft lignin.

According to another embodiment of the present invention, the cosmetic formulation is a sunscreen product, facial makeup product, hair care product, hair styling product, nail care product, hand care product, skin care product and mixtures thereof.

The scope and interest of the invention will be better understood based on the following examples which are intended to illustrate certain embodiments of the present invention and are non-limitative.

1. A dry composition for chemical and physical sun protection, the composition comprising
a) at least one calcium carbonate, and
b) from 0.1 wt.-% to 100 wt.-%, based on the dry weight of the at least one calcium carbonate of step a) of at least one lignin.

2. The composition according to claim 1, wherein the calcium carbonate is selected from the group consisting of ground calcium carbonate (GCC), preferably marble, limestone, chalk, precipitated calcium carbonate (PCC), preferably vaterite, calcite and/or aragonite, and mixtures thereof and most preferably the calcium carbonate is ground calcium carbonate.

3. The composition according to claim 1, wherein the at least one calcium carbonate has
a) a weight median particle size d₅₀ value in the range from 0.05 μm to 20 μm, preferably from 0.25 μm to 10 μm and most preferably from 0.5 μm to 8 μm, and/or
b) a top cut (d₉₈) of ≤100 μm, preferably ≤60 μm, more preferably ≤45 μm and most preferably ≤20 μm, and/or
c) a specific surface area (BET) of from 0.5 to 100 m²/g, preferably from 0.5 to 50 m²/g, more preferably from 0.5 to 35 m²/g and most preferably from 0.5 to 10 m²/g as measured by the BET nitrogen method.

4. The composition according to claim 1, wherein the at least one lignin is a water soluble or water insoluble lignin selected from the group consisting of natural lignin, klason lignin, hydrolyzed lignin, milled wood lignin, soda lignin, organosolv lignin, kraft lignin, sulphonated lignin and mixtures thereof, preferably is a water-insoluble lignin selected from the group consisting of klason lignin, kraft lignin and mixtures thereof and most preferably is kraft lignin.

5. The composition according to claim 1, wherein the at least one lignin is present in the composition in an amount from 1 to 50 wt.-%, based on the dry weight of the at least one calcium carbonate of step a), preferably in an amount of 3 to 30 wt.-% and most preferably in an amount of 5 to 25 wt.-%.

6. The composition according to claim 1, wherein the composition further comprises an organic solvent, preferably in an amount of 5 to 50 wt.-%, based on the dry weight of the at least one calcium carbonate of step a), more preferably in an amount of 10 to 40 wt.-% and most preferably in an amount of 15 to 35 wt.-% and/or preferably in an amount of 100 to 500 wt.-%, based on the dry weight of the at least one lignin of step b), more preferably in an amount of 150 to 450 wt.-% and most preferably in an amount of 200 to 300 wt.-%.

7. The composition according to claim 6, wherein the organic solvent is selected from the group consisting of hexane, toluene, methanol, ethanol, dioxane, acetone, dimethyl sulfoxide, dimethylformamide, ethylene glycol, ethylacetate, glycerol, γ-valerolactone, polyethylene glycol, polypropylene glycol and mixtures thereof, more preferably is selected from the group consisting of ethylene glycol, ethylacetate, glycerol, γ-valerolactone, polyethylene glycol, polypropylene glycol and mixtures thereof, and most preferably is γ-valerolactone.

8. The composition according to claim 1, wherein the at least one lignin is present in the composition in the form of a mixture with or a coating on the at least one calcium carbonate, and preferably is present as a coating on the at least one calcium carbonate.

9. The composition according to claim 8, wherein the at least one lignin in the coating on the at least one calcium carbonate is a water insoluble lignin, and preferably kraft lignin.

10. A fluid composition for chemical and physical sun protection, the fluid composition comprising water and 1 to 100 wt.-% of the dry composition according to claim 1, based on the weight of the water.

11. The composition according to claim 10, wherein the composition further comprises a surfactant,
preferably selected from the group consisting of monomers and/or co-monomers of acrylic acid, methacrylic acid, itaconic acid, crotonic acid, fumaric acid, maleic anhydride acid, isocrotonic acid, aconitic acid (cis or trans), mesaconic acid, sinapinic acid, undecylenic acid, angelic acid, canellic acid, hydroxyacrylic acid, acrolein, acrylamide, acrylonitrile, dimethylaminoethyl methacrylate, vinylpyrrolidone, vinylcaprolactam, ethylene, propylene, isobutylene, diisobutylene, vinyl acetate, styrene, α-methyl styrene, methyl vinyl ketone, the esters of acrylic and methacrylic acids, organo modified trisiloxane, PEG, polyglycerol-ester, sophorolipid, polyether and mixtures thereof, and most preferably is poly(acrylic acid) and/or poly (methacrylic acid) and/or,
wherein the surfactant is present in the composition in an amount of 0.0001 to 0.2 wt.-%, based on the dry weight of the at least one calcium carbonate of step a) and the at least one lignin of step b), preferably in an amount of 0.001 to 0.1 wt.-% and most preferably in an amount of 0.005 to 0.05 wt.-%.

12. An emulsion for chemical and physical sun protection, the emulsion comprising a water in oil or oil in water mixture and 0.1 to 100 wt.-% of the dry composition according to claim 1, based on the weight of the water in oil or oil in water mixture.

13. A method of using the composition according to claim 1 for sun protection of plants and parts thereof, comprising the step of:
applying the composition to plants and parts thereof,
wherein the sun protection includes physical and chemical protection.

14. The method according to claim 13, wherein the plants and parts thereof are fruits, vegetables, trees, seeds, leaves, wood, nuts, crops, crop plants and flowers.

15. A method of using the emulsion of claim 12 for chemical and physical sun protection in a cosmetic formulation, comprising the step of adding the emulsion to the cosmetic formulation.

16. The method according to claim 15, wherein the at least one lignin is present in the form of a coating on the at least one calcium carbonate and/or wherein the at least one lignin is a water-insoluble lignin, preferably kraft lignin.

17. The method according to claim 15, wherein the cosmetic formulation is a sunscreen product, facial makeup product, hair care product, hair styling product, nail care product, hand care product, skin care product and mixtures thereof.

18. The composition according to claim 3, wherein the at least one lignin is present in the composition in the form of a mixture with or a coating on the at least one calcium carbonate, and preferably is present as a coating on the at least one calcium carbonate.

19. The composition according to claim 18, wherein the at least one lignin in the coating on the at least one calcium carbonate is a water insoluble lignin, and preferably kraft lignin.

20. The composition according to claim 3, wherein the at least one lignin is present in the composition in an amount from 1 to 50 wt.-%, based on the dry weight of the at least one calcium carbonate of step a), preferably in an amount of 3 to 30 wt.-% and most preferably in an amount of 5 to 25 wt.-%.
